(19) United States
(12) Patent Application Publication
(10) Pub. No.: US 2017/0326189 A1
(43) Pub. Date: Nov. 16, 2017

(54) Process for Obtaining a Composition Enriched with Dihydroquercetin or with Tannins

(71) Applicant: Institut National de la Recherche Agronomiquye (INRA), Paris cedex 07 (FR)

(72) Inventors: Chahinez Aouf, Montpellier (FR); Claire Mayer, Montpellier (FR); Abdellatif Barakat, Montpellier (FR); Helene Hoh, Saint Gely Du Fesc (FR)

(21) Appl. No.: 15/535,602

(22) PCT Filed: Dec. 15, 2015
(86) PCT No.: PCT/EP2015/079684

§ 371 Date: Jun. 13, 2017

(30) Foreign Application Priority Data
Dec. 15, 2014 (FR) 1462419

(51) Int. Cl. A61K 36/15; B02C 23/08; A61K 9/14; A61K 31/352

(52) U.S. Cl. CPC A61K 36/15; A61K 31/352; B02C 23/08; A61K 9/14; A61K 2236/17; A61K 2236/15

(57) ABSTRACT

The invention relates to a method for obtaining a composition based on conifer bark enriched in dihydroquercetin or in tannins, comprising the following steps:
a) grinding dried shavings of bark from a conifer to obtain a ground particulate material of conifer bark, and
b) selecting, from the ground particulate material obtained in step a), the fraction of particles:
   of smallest size representing at most 40% by weight, relative to the total weight of the ground particulate material obtained in step a), this fraction being enriched in tannins; or
   of largest size representing at most 50% by weight, relative to the total weight of the ground particulate material obtained in step a), this fraction being enriched in dihydroquercetin.

### FIELD OF THE INVENTION

The present invention relates to the field of obtaining compositions enriched in certain polyphenols, respectively dihydroquercetin or tannins.

### PRIOR ART

Polyphenols, or phenolic compounds, are organic molecules which are products of the secondary metabolism of plants. They are characterized chemically by the presence of several phenol groups.

It has been shown that polyphenols have beneficial effects on human health. Indeed, their properties of natural antioxidants could play a part in preventing and treating cancer, and inflammatory, cardiovascular and neurodegenerative diseases. These chemical compounds are also used as additives in the food processing, pharmaceutical and cosmetics industries.

Among the polyphenols, the family of the flavonoids comprises more than 6000 compounds, subsumed into 4 main groups: flavones, flavonones and dihydroflavonones (flavanonols), flavanols and anthocyanins.

This large family comprises both monomeric compounds and polymeric compounds.

Tannins are polymeric compounds. There are 2 main categories of tannins: catechin tannins, also referred to as condensed tannins or procyanidins, and hydrolysable tannins, predominantly comprising gallotannins and ellagitannins. In terms of chemistry, they consist of either oligomers or polymers of flavanols (condensed tannins), or of polyols to which galloyl units or derivatives thereof are attached (hydrolysable tannins).

Tannins are naturally present in numerous plants, especially grapes. They have been used for centuries in tanning hides, because they have the natural property of causing proteins to precipitate. They are also beneficial for their capacity to chelate metal ions and especially heavy cations. In oenology, tannins are used for clarification of wines because they have a strong aptitude to combine with proteins. Applications for tannins extend to numerous other fields, such as those of food processing, pharmacy, cosmetics, ceramics, and textiles.

Dihydroquercetin, also referred to as taxifolin, is a monomeric organic compound from the flavanonol family. Flavanonols (3-hydroxyflavanone or 2,3-dihydroflavonol) are a class of flavonoids derived from 3-hydroxy-2,3-dihydro-2-phenylchromen-4-one.

Taxifolin is naturally present in acai (Euterpe oleracea), Siberian larch (Latrix sibirica) and silymarin extracted from milk thistle seeds. Taxifolin is mainly produced by extraction in Russia.

Taxifolin has numerous properties which are potentially beneficial for health. It has especially been shown in vitro that taxifolin protects cell membranes, and improves capillary activity and blood microcirculation. It has also been shown that taxifolin has anti-inflammatory, anti-allergenic or else hepatoprotective activities. It is also known that the antioxidant properties of taxifolin are superior to those of the other flavonoids, or even those of vitamin E. Moreover, taxifolin does not exert any mutagenic effect and has greatly reduced toxicity properties. In particular, taxifolin is even less toxic than the compound quercetin, to which it is related.

Due to its numerous beneficial properties for health, taxifolin is widely used in the pharmaceutical field, in the food industry and in the cosmetics field.

In the pharmaceutical field, taxifolin may be prescribed especially in complex treatments for vitamin deficiency, cardiovascular diseases, ischemic cardiopathy and atherosclerosis. Some studies encourage the use of taxifolin for cancer treatment. Regarding the medical properties of taxifolin, those skilled in the art may advantageously refer to the paper by Weidmann (2012, European Journal of Pharmacology, Vol. 684: 19-26).

In the food industry, taxifolin is used as an antioxidant, in particular in the manufacture of food products containing fatty substances. Taxifolin is especially used in the preparation of products based on meat or fish, dairy products or else of confectionery.

There is thus an increasing demand from manufacturers for large quantities of taxifolin to be available. Despite this, current production of taxifolin is insufficient to satisfy the needs of the market. Consequently, taxifolin is sold at very high prices.

Virtually all of the methods for obtaining taxifolin consist of methods for aqueous-alcoholic extraction at high temperature, the extraction steps themselves being followed by one or more steps of purifying the taxifolin contained in the extracts. The taxifolin extracts are mainly purified by chromatography or by re-crystallization. The extraction of tannins is also carried out by using one or more solvents such as acetone and ethyl acetate. It may be followed by a step of depolymerization, a routine analytical method which relies on the acid-catalyzed breakage of intermonomeric bonds.

An example of these known methods for extracting polyphenols by solvent, in this case on the laboratory scale, is illustrated by the paper from Bocalandro et al. (2012, Industrial Crops and Products, Vol. 38: 21-26). Bocalandro et al. describe a method in which bark shavings from Pinus radiata are subjected to a step of aqueous-alcoholic extraction at 120° C. The extract obtained in this way was then subjected to a step of evaporation in order to eliminate the residual ethanol. The resultant aqueous extract is filtered in order to separate the water-soluble polyphenol fraction and the non-water-soluble polyphenol fraction. The water-soluble polyphenol fraction is then lyophilized and then stored in sealed containers. Within the polyphenols which have been extracted, taxifolin is present at an amount of approximately 17 mg per gram of extract. Tannins are present at an amount of approximately 330 to 340 mg per gram of extract.

An example of obtaining taxifolin which uses a step of aqueous-alcoholic extraction is described in European patent application no. EP 2 639 232. After eliminating the trunk tissues, Siberian larch wood is fragmented and dried at a temperature of 40-50° C. until a residual humidity of 15-25% is obtained. The dried wood is then ground, and then the resulting sawdust is subjected to a step of aqueous-alcoholic extraction at a temperature of 40-50° C. The agent of aqueous-alcoholic extraction is then distilled and the sawdust is subjected to mechanical pressing in order to recover the residual alcohol. The aqueous part of the extract is then cooled for the purpose of eliminating resinous impurities. The aqueous portion of the extract with resinous impurities removed is subjected to a step of chromatography and taxifolin is recovered in one or more fractions of the eluate, depending on the desired degree of taxifolin purity. After evaporation of the residual ethanol, the elution product is then crystallized.

Another method for obtaining taxifolin by solvent extraction is described in the PCT application no. WO 2013/137844. According to another method, described by Wang et al. (2011, Food Chemistry, Vol. 126: 1178-1185), the step of extracting polyphenols is carried out, starting from sawdust, by means of an aqueous solution containing a combination of enzymes, respectively a cellulase, a pectinase and a beta-glucosidase. Nonetheless, the method of Wang et al. is analytical and does not comprise a step of preparative separation of taxifolin from the other polyphenols contained in the enzymatic extract.

These methods for extraction starting from ground conifer barks do not include a selection of the bark particles as a function of their size, and no fraction enriched in dihydroquercetin and/or in tannins has been identified.

The result of the above is that the known methods for obtaining taxifolin and tannins comprise a plurality of steps of pressing and of intense washing with water or organic solvents. The use of these methods for obtaining taxifolin and tannins is expensive, especially due to the large volumes of solvents required and the energy costs. Moreover, carrying out repeated steps of extraction and separation may contribute to degrading the phenolic compounds, especially taxifolin, since this compound rapidly oxidizes on contact with air.

Due to the abovementioned technical drawbacks, the known methods for extracting taxifolin have a low yield.

Similarly, there are no “dry” methods for extracting tannins, based on pre-selection of a fraction of bark enriched in tannins. There is a requirement in the prior art to make available methods for obtaining taxifolin and tannins which are alternative or improved compared to the known methods.

### SUMMARY OF THE INVENTION

The invention relates to a method for obtaining a composition based on conifer bark enriched in dihydroquercetin and/or in tannins, comprising the following steps:

a) grinding dried shavings of bark from a conifer to obtain a ground particulate material of conifer bark, and

b) selecting, from the ground particulate material obtained in step a), the fraction of bark particles:
of largest size representing at most 50% by weight, relative to the total weight of the ground particulate material obtained in step a), this fraction being enriched in dihydroquercetin; or
   of smallest size representing at most 40% by weight, relative to the total weight of the ground particulate material obtained in step a), this fraction being enriched in tannins.

According to a first aspect, the present invention relates to a method for obtaining a composition enriched in tannins, comprising steps of enrichment in tannins which are carried out under dry conditions, without requiring the use of solvent.

The present invention thus relates to a method for obtaining a composition based on conifer bark enriched in tannins, comprising the following steps:

a) grinding dried shavings of bark from a conifer to obtain a ground particulate material of conifer bark, and
b) selecting, from the ground particulate material obtained in step a), the fraction of particles of smallest size representing at most 40% by weight, relative to the total weight of the ground particulate material obtained in step a).

According to a variant of the above method, said method comprises the following steps:

a) grinding dried shavings of bark from a conifer to obtain a ground particulate material of conifer bark,

b1) separating, depending on their size, the bark particles contained in the ground material obtained in step a) into a plurality of fractions comprising particles of distinct sizes, and

b2) selecting a fraction of bark particles enriched in tannins, said fraction consisting of bark particles having a size less than 0.30 mm, and preferentially of bark particles having a size less than 0.16 mm.

In some embodiments of the above method, said conifer is a maritime pine.

The invention also relates to a fraction of bark particles enriched in tannins, obtainable by the above method.

The invention also relates to a composition enriched in tannins, obtainable by the above method.

According to a second aspect, the present invention relates to a method for obtaining a composition enriched in taxifolin, comprising steps of enrichment in taxifolin which are carried out under dry conditions, without requiring the use of solvent.

The present invention thus relates to a method for obtaining a composition based on conifer bark enriched in dihydroquercetin, comprising the following steps:

a) grinding dried shavings of bark from a conifer to obtain a ground particulate material of conifer bark, and

b) selecting, from the ground particulate material obtained in step a), the fraction of particles of largest size representing at most 50% by weight, relative to the total weight of the ground particulate material obtained in step a).

According to a variant of the above method, said method comprises the following steps:

a) grinding dried shavings of bark from a conifer to obtain a ground particulate material of conifer bark,

b1) separating, depending on their size, the bark particles contained in the ground material obtained in step a) into a plurality of fractions comprising particles of distinct sizes, and

b2) selecting a fraction of bark particles enriched in dihydroquercetin, said fraction consisting of bark particles having a size greater than 0.6 mm, and preferentially of bark particles having a size greater than 1 mm.

In some embodiments of the above methods, the dried shavings used in step a) have a size of the order of a few cm.

In some embodiments of the above methods, step a) of grinding is carried out according to a method selected from (1) grinding in a knife mill, (2) grinding in a ball mill, and (3) grinding in a knife mill followed by grinding in a jet mill.

Nonetheless, as is specified below in the description, when a jet mill is used in step a), it is preferable to carry out, in step a), a pre-grinding of the shavings by means of a knife mill then to subject the coarse ground material obtained in this way to fine grinding by means of a jet mill.

In some embodiments of the above methods, the latter also comprise a step c) of solvent extraction of the tannins or the dihydroquercetin contained in the particles of the fraction enriched in tannins or dihydroquercetin obtained in step b), or b2).

In some embodiments of the above method, said conifer is a Douglas pine.

The invention also relates to a fraction of bark particles enriched in dihydroquercetin, obtainable by the above method.

The invention also relates to a composition enriched in dihydroquercetin, obtainable by the above method.

### DESCRIPTION OF THE FIGURES

FIG. 1: Percentage by weight of each fraction of bark particles F1 (particle size greater than 1 mm), F2 (particle size less than 1 mm and greater than 0.56 mm), F3 (particle size less than 0.56 mm and greater than 0.16 mm), and F4 (particle size less than 0.16 mm). Y-axis: percentage relative to total weight of the starting sample of bark particles (fraction F0).

### DETAILED DESCRIPTION OF THE INVENTION

The present invention provides an improved method for obtaining a composition enriched in certain polyphenols, and more particularly a composition enriched in dihydroquercetin and a composition enriched in tannins.

It is shown according to the invention that, unexpectedly, a composition enriched in dihydroquercetin may be obtained from a particular fraction of the tissues forming the bark of a conifer. The inventors have especially shown that some tissues which constitute the bark of a conifer, in particular the Douglas pine, are particularly rich in dihydroquercetin. The inventors have also shown that all these conifer bark tissues having a high content of dihydroquercetin contain a large portion of the total amount of dihydroquercetin contained in the bark of these trees.

Moreover, the inventors have also shown that other tissues which constitute the bark of a conifer were particularly rich in other polyphenols, such as tannins.

The applicant has developed a method specifically adapted to obtaining fractions of the bark tissues which have been identified as having a high content of certain polyphenols, and in particular respectively the fraction of bark tissue having a high content of dihydroquercetin or the fraction of bark tissue having a high content of tannins.

Unexpectedly, the applicant has shown that the fractions of bark tissues of interest may be separated from the other fractions of bark tissues by grinding, then separation, depending on their size, of the bark particles contained in the resulting ground material.

The inventors have thus shown that the dihydroquercetin present in conifer bark is mainly located in some of the tissues which constitute this bark. It emerges from the results obtained by the inventors that a composition enriched in dihydroquercetin may be obtained from conifer bark by means of a method which would make it possible to obtain a product enriched in the tissue(s) in which the dihydroquercetin is mainly located.

Such a method, adapted for obtaining a composition based on conifer bark enriched in dihydroquercetin is provided according to the invention.

The inventors have also shown that the tannins present in conifer bark are mainly located in some of the tissues which constitute this bark. It emerges from the results obtained by the inventors that a composition enriched in tannins may be obtained from conifer bark by means of a method which would make it possible to obtain a product enriched in the tissue(s) in which the tannins are mainly located.

According to a first aspect, the present invention relates to a method for obtaining a composition based on conifer bark enriched in tannins, comprising the following steps:
a) grinding dried shavings of bark from a conifer to obtain a ground particulate material of conifer bark, and
b) selecting, from the ground particulate material obtained in step a), the fraction of bark particles of smallest size representing at most 40% by weight, relative to the total weight of the ground particulate material obtained in step a).

According to a variant of the above method, said method comprises the following steps:
a) grinding dried shavings of bark from a conifer to obtain a ground particulate material of conifer bark,
b1) separating, depending on their size, the bark particles contained in the ground material obtained in step a) into a plurality of fractions comprising particles of distinct sizes, and
b2) selecting a fraction of bark particles enriched in tannins, said fraction consisting of bark particles having a size less than 0.30 mm, and preferentially of bark particles having a size less than 0.16 mm.

According to a second aspect, the present invention relates to a method for obtaining a composition based on conifer bark enriched in dihydroquercetin, comprising the following steps:
a) grinding dried shavings of bark from a conifer to obtain a ground particulate material of conifer bark, and
b) selecting, from the ground particulate material obtained in step a), the fraction of particles of largest size representing at most 50% by weight, relative to the total weight of the ground particulate material obtained in step a).

In these two methods, step a) of grinding may be carried out by any technique known to those skilled in the art. It may especially be carried out, according to known techniques, using a grinding device selected from (I) a knife mill, (2) a ball mill, and (3) a jet mill. In some embodiments of step a) of grinding, a combination of several of these devices is used.

Step b) for selecting the fraction of particles:
either of smallest size representing at most 40% by weight, relative to the total weight of the ground particulate material obtained in step a),
or of largest size representing at most 50% by weight, relative to the total weight of the ground particulate material obtained in step a),

may be carried out according to any technique known for separating particles depending on their size.

By way of illustration, step b) may be carried out by sieving, using a series of sieves with successively smaller mesh sizes.

In this embodiment, (i) the weight of the ground particulate material obtained in step a) is determined, then (ii) the sieving operation is carried out, which leads to the separation of the particles of said ground material into a plurality of fractions of particles of distinct sizes, the weight of each fraction of particles also being determined, then (iii) the fraction(s) comprising the smallest and/or largest particles are selected, it being understood that the weight of the fraction of particles selected, or the weight of the plurality of fractions selected, represents at most 40% or 50% of the total weight of the ground particulate material obtained in step a).

As is shown in the examples, the particles of the ground material obtained in step a) which are the most enriched in tannins are those of the smallest size. Consequently, the fraction of particles is selected in step b) especially according to the desired degree of enrichment in tannins of the final composition. When a moderate enrichment in tannins is desired for the final composition, it is possible to select, in step b), the fraction of particles of the smallest size which represents at most 40% of the total weight of the ground particulate material obtained in step a). In other situations in which a greater degree of enrichment in tannins is sought for the final composition, it is possible to select, in step b), the fraction of particles of the smallest size which represents at most 30% or 20% of the total weight of the ground particulate material obtained in step a).

As is shown in the examples, the particles of the ground material obtained in step a) which are the most enriched in dihydroquercetin are those of the largest size. Consequently, the fraction of particles is selected in step b) especially according to the desired degree of enrichment in dihydroquercetin of the final composition. When a moderate enrichment in dihydroquercetin is desired for the final composition, it is possible to select, in step b), the fraction of particles of the largest size which represents at most 50% of the total weight of the ground particulate material obtained in step a). In other situations in which a greater degree of enrichment in dihydroquercetin is sought for the final composition, it is possible to select, in step b), the fraction of particles of the largest size which represents at most 40%, 30% or 20% of the total weight of the ground particulate material obtained in step a).

For the purposes of the present description, a fraction of particles representing at most 40% by weight, relative to the total weight of the ground particulate material obtained in step a), encompasses a fraction of particles representing at most 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16% and 15% by weight relative to the total weight of the ground particulate material obtained in step a).

For the purposes of the present description, a fraction of particles representing at most 50% by weight, relative to the total weight of the ground particulate material obtained in step a), encompasses a fraction of particles representing at most 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21% and 20% by weight relative to the total weight of the ground particulate material obtained in step a).

Without wishing to be bound by any particular theory, the applicant believes that the selection of a fraction of particles representing more than 50% by weight, relative to the total weight of the ground material obtained in step a), leads to obtaining a final composition which is insufficiently enriched in dihydroquercetin for the purposes of industrial use.

In addition, the applicant is also of the opinion that the selection of a fraction of particles representing less than 20% by weight relative to the total weight of the ground material obtained in step a), leads to obtaining a final composition which is substantially enriched in dihydroquercetin, but too much of the dihydroquercetin contained initially in the starting product is thus not used.

By way of illustration, for the two methods according to the invention, step a) of grinding may be carried out by means of a knife mill, for example by means of a knife mill fitted with a 6 mm outlet grating.

According to a preferred aspect, in step b), the following are selected by sieving: either:
a fraction of bark particles enriched in tannins, consisting of bark particles having a size of less than 0.30 mm, preferentially less than 0.16 mm; or
a fraction of particles having a particle size of greater than 1 mm.

In the embodiment of the method which is illustrated in the examples, the fraction of particles having a size of less than 0.16 mm represents approximately 16% by weight relative to the total weight of the ground particulate material obtained in step a).

In the embodiment of the method which is illustrated in the examples, the fraction of particles having a particle size of greater than 1 mm represents approximately 26% by weight relative to the total weight of the ground particulate material obtained in step a).

The method according to the invention makes it possible to obtain compositions enriched in polyphenols, in particular in tannins or dihydroquercetin, by means of physical steps of enrichment, carried out at room temperature and not using a solvent, thereby avoiding degradation of the polyphenols of interest, specifically taxifolin. Due to the fact that they do not require heating or solvent, the steps of enrichment in polyphenols, specifically in tannins or in taxifolin, of the above method, are less costly than the known steps for solvent extraction, which are carried out at high temperature.

In some embodiments of the method for obtaining a composition based on conifer bark enriched in dihydroquercetin, said method comprises the following steps:
a) grinding dried shavings of bark from a conifer to obtain a ground particulate material of conifer bark,
b1) separating, depending on their size, the bark particles contained in the ground material obtained in step a) into a plurality of fractions comprising particles of distinct sizes, and
b2) selecting a fraction of bark particles enriched in dihydroquercetin, said fraction consisting of bark particles having a size greater than 0.6 mm, and preferentially of bark particles having a size greater than 1 mm.

Thus, in the above variant of the method according to the invention, step b) in turn comprises the specified steps b1) and b2).

“Bark shavings” are intended to mean fragments of bark, without any distinction in terms of size or shape. In general, in their longest length, the bark shavings have a size which may vary from a few centimeters to a few tens of centimeters, for example from 5 cm to 30 cm. The thickness of the shavings generally varies from a few millimeters to a few centimeters, for example from 1 mm to 5 cm, and often from 1 mm to 3 cm.

“Tannins” are intended to mean condensed tannins, such as flavonoid polymers, and hydrolysable tannins, such as esters of gallic acid or of ellagic acid.

The term tannins preferentially refers to flavonoid polymers.

According to the invention, “composition enriched in tannins” is intended to mean a composition obtained from a starting material and having a content of tannins which is substantially higher than the content of tannins in said starting material, in particular having a content of tannins which is at least double the content of tannins in the starting material. For the purposes of the present description, a composition enriched in tannins encompasses the compositions resulting from the method, the content of tannins of which is at least 20% greater than the content of tannins of the starting product, namely the dried conifer bark shavings. It is specified that, for the purposes of the present description, two values for content of tannins are compared after taking into account the degree of humidity of the respective materials compared.

“Dihydroquercetin” is intended to mean the compound (2R,3R)-2-(3,4-dihydroxyphenyl)3,5,7-trihydroxy-2-3-dihydrochromen-4-one, which is also commonly denoted “taxifolin”, and the CAS reference of which is CAS 480-18-2.

According to the invention, “composition enriched in dihydroquercetin” is intended to mean a composition obtained from a starting material and having a content of dihydroquercetin which is substantially higher than the content of dihydroquercetin in said starting material, in particular having a content of dihydroquercetin which is at least double the content of dihydroquercetin in the starting material. For the purposes of the present description, a composition enriched in dihydroquercetin encompasses the compositions resulting from the method, the content of dihydroquercetin of which is at least 20% greater than the content of dihydroquercetin of the starting product, namely the dried conifer bark shavings. It is specified that, for the purposes of the present description, two values for content of dihydroquercetin are compared after taking into account the degree of humidity of the respective materials compared.

For the purposes of the present description, the size of particles is preferentially determined according to a sieving method, for which a succession of sieves with increasingly smaller mesh sizes is used. By way of illustration, use may be made of a succession of sieves, the mesh size of which is respectively 10.0 mm, 6.0 mm, 4.0 mm, 3.0 mm, 2.0 mm, 1.0 mm, 0.50 mm, 0.40 mm, 0.30 mm, 0.25 mm, 0.20 mm, 0.15 mm, and 0.10 mm.

Step a) of Grinding

In step a), dried shavings of conifer bark are ground in order to obtain a set of bark particles of small size, able to undergo a second grinding step.

The ground material obtained at the end of step a) preferably comprises a set of particles having particle size characteristics such that these particles have a particle size of greater than 1 μm.

The ground material obtained at the end of step a) preferably comprises particles having a size equal to or less than 10 mm, and most preferably a size equal to or less than 6 mm.

The particle size profile of the particles contained in the ground material may advantageously be homogeneous or heterogeneous, with a particle size which may range up to 6 mm.

The bark particles resulting from step a) of grinding are collected for carrying out the rest of the method.

As indicated above, step a) is carried out with dried shavings. The inventors have shown that using very wet shavings, that is to say shavings for which the moisture content is greater than 50%, in step a), causes clogging of the grinding device. Clogging of the grinding device is especially illustrated by clogging of the selection grating, which prevents the particles from leaving the mill and being collected for the remainder of the method. In particular, the particles remain in the chamber of the grinding device in an undesired manner, which results in a ground material which is difficult to collect at the end of step a) and the particles are thus unsuitable for carrying out the rest of the method.

Advantageously, before step a), the shavings are subjected to a step of drying at a temperature ranging from 30° C. to 40° C. for a duration ranging from 6 hours to 36 hours, better still from 10 hours to 30 hours, advantageously for approximately 24 hours. Those skilled in the art will know to adapt the temperature and the duration of the method depending on the degree of moisture of the starting shavings.

A drying temperature below 30° C. is ineffective. A drying temperature of greater than 40° C. should be avoided, in order not to degrade the polyphenols contained in the bark, and in particular not to degrade the taxifolin or the tannins contained in the bark.

Advantageously, the dried shavings used in step a) have a residual moisture content of equal to or less than 40%, preferably a residual moisture content of equal to or less than 15%.

For the purposes of the present description, the residual moisture content is determined by the gravimetric method according to the following indications: the residual moisture is removed from the product tested by heating. The residual moisture content of the product tested is calculated as a function of the loss of weight of the product over the course of the drying cycle. The residual moisture content is calculated according to the conventional technique consisting in (i) carrying out a first weighing of a set of bark shavings, (ii) drying said set of bark shavings, for example by passage in an oven at 120° C., for example for 24 hours, for example at reduced pressure, (iii) carrying out a second weighing of said set of bark shavings after drying, and (iv) calculating the initial water content (residual moisture) of said set of bark shavings from the calculation of the amount of evaporated water.

In order to carry out the two methods described, step a) may be carried out using any grinding device making it possible to obtain, from bark shavings, bark particles having the particle size specified in the present description. Those skilled in the art may especially use a grinding device selected from (1) grinding in a knife mill, (2) grinding in a ball mill, and (3) grinding in a jet mill, and, where appropriate, a combination of several of these devices, that is to say at least two of these devices. Thus, step a) of grinding may be carried out according to a method selected from (1) grinding in a knife mill, (2) grinding in a ball mill, and (3) grinding in a jet mill, or else according to a combination of at least two of these methods.

In some embodiments, step a) is carried out using a knife mill of known type. Those skilled in the art may for example use a knife mill of Retsch SM100 type, sold by Retsch.

Step a) of grinding is advantageously carried out at a rotor speed of approximately 1500 revolutions per minute. It is specified that when a knife mill is used in step a), the grinding is preferentially carried out continuously. Thus, the ground particulate material is generated continuously in step a) before separation of the particles constituting the ground material in step b). With this type of mill, the duration of the grinding step is not critical, since the maximum size of the particles contained in the ground material is determined by the mesh size of the selection grating, at the outlet of the mill. By way of illustration, the mill is fitted with an outlet grating of 6 mm if the desired maximum size of the particles constituting the ground material obtained at the end of step a) is 6 mm.

In other embodiments, step a) is carried out using a ball mill, also known as a bead mill, of known type. Those skilled in the art may for example use a ball mill sold by Faure (France). By way of illustration, a ball mill may be used, into which balls of different diameters have been introduced, respectively balls of 1.2 cm, 1.6 cm and 2.6 cm in diameter. For example, it is possible to introduce balls of 1.2 cm, 1.6 cm and 2.6 cm into the chamber of the mill in the proportions ⅓, ⅓ and ⅓.

Alternatively, in these embodiments, step a) comprises two steps of grinding, respectively the following steps:

a1) a first step comprising grinding dried conifer bark shavings using a knife mill, in order to obtain a ground material of shavings, and

a2) a second step comprising grinding the ground material of shavings obtained in step a1) using a ball mill, in order to obtain a ground particulate material of conifer bark.

With a ball mill, step a) of grinding is preferentially carried out at 70% of the critical speed. It is specified that the critical speed is defined as the speed at which the balls are located on the walls of the chamber due to centrifugal force. It is understood that the critical speed depends especially on the size and density of the balls used and also on the diameter of the chamber of the grinding device.

By way of illustration, when step a) is carried out with a ball mill, step a) may be carried out for a duration which may vary from 10 minutes to 120 hours, for example applying a speed of rotation of the chamber of approximately 120 revolutions per minute.

In yet other embodiments, step a) is carried out using a jet mill of known type. Those skilled in the art may for example use a jet mill sold by Hosokawa-Alpine. By way of illustration, a jet mill may be used at a pressure of 4.5 bar and a speed of rotation of the classifying rotor of approximately 2000 revolutions per minute.

According to a first alternative, in these embodiments, step a) comprises two steps of grinding, respectively the following steps:

a1) a first step comprising grinding dried conifer bark shavings using a knife mill, in order to obtain a ground material of shavings, and

a2) a second step comprising grinding the ground material of shavings obtained in step a1) using a jet mill, in order to obtain a ground particulate material of conifer bark.

According to a second alternative, in these embodiments, step a) comprises two steps of grinding, respectively the following steps:

a1) a first step comprising grinding dried conifer bark shavings using a knife mill fitted with a 6 mm selection grating, in order to obtain a first ground material of shavings,

a2) a second step comprising grinding the ground material of dried shavings obtained in step a1) using a knife mill fitted with a 1 mm selection grating, in order to obtain a second ground material of shavings,

a2) a third step comprising grinding the second ground material of shavings obtained in step a2) using a jet mill, in order to obtain a ground particulate material of conifer bark.

Step b) of Selection or Steps b1) and b2) of Separation then Selection

Step b), or step b1) in some embodiments of the method, comprises the separation of the bark particles obtained in step a) in order to obtain a plurality of fractions comprising particles of distinct sizes.

In some embodiments of the method, the operating conditions of step b), or b1), are adapted to obtain a plurality of fractions of bark particles, respectively at least:

(i) a fraction of bark particles having a particle size of equal to or greater than 0.60 mm, for example a particle size equal to or greater than 1.00 mm, and

(ii) a fraction of bark particles having a particle size of less than 1.00 mm, for example a particle size of less than 0.60 mm.

In some embodiments of the method, the operating conditions of step b), or b1), are adapted to obtain a plurality of fractions of bark particles, respectively at least:

(i) a fraction of bark particles having a particle size of equal to or greater than 0.60 mm, for example a particle size equal to or greater than 1.00 mm, and

(ii) a fraction of bark particles having a particle size of equal to or less than 0.30 mm, for example a particle size of equal to or less than 0.16 mm.

For the purposes of the present description, a particle size of greater than 0.60 mm encompasses a particle size of greater than 0.65 mm, 0.70 mm, 0.75 mm, 0.80 mm, 0.90 mm, 0.95 mm, 1.00 mm, 1.05 mm and 1.10 mm, 1.15 mm, 1.20 mm, 1.25 mm and 1.30 mm.

For the purposes of the present description, a particle size of less than 1.00 mm encompasses a particle size of less than 0.90 mm, 0.80 mm, 0.70 mm and 0.65 mm.

For the purposes of the present description, a particle size of equal to or less than 0.30 mm encompasses a particle size of less than 0.25 mm, 0.20 mm and 0.16 mm.

In some embodiments of step b) of separation, or of step b1) in some variants of the method, other fractions of bark particles are obtained, which are in addition to the fractions (i) and (ii) above, for example fractions, the particle size of which is intermediate between the fractions (i) and (ii) above, as is illustrated in the examples.

In some embodiments, step b), or b1), of separation of the bark particles is carried out by sieving. By way of illustration, the bark particles obtained in step a) are separated in step b) using, successively:
a first sieve with a mesh size which may vary from 0.60 mm to 1.30 mm, for example a sieve with a mesh size of 1.00 mm, for the purposes of obtaining the fraction (i) of bark particles above, which is retained by the sieve, and
a second sieve with a mesh size which may vary from 0.10 mm to 0.30 mm, for example a sieve with a mesh size of 0.16 mm, for the purposes of obtaining the fraction (ii) of bark particles above, which is not retained by the sieve.

In other embodiments, additional sieves are used in step b), the mesh size of which is intermediate between the mesh sizes of the first and second sieves, respectively, described above, as is illustrated in the examples.

Carrying Out the Rest of Step b) or Carrying Out Step b2) of Selection

In step b), or in step b2) in some variants of the methods, the fraction(s) of conifer bark particles which are enriched in tannins or in dihydroquercetin are selected, that is to say those in which the content of tannins or of dihydroquercetin is substantially higher than the content of tannins or of dihydroquercetin in the conifer bark shavings used as starting material.

Preferentially, in step b), or in step b2), the fraction comprising bark particles having a particle size equal to or greater than a particle size ranging from 0.6 mm to 1.00 mm is selected, and preferentially bark particles having a size greater than 1.00 mm. As is shown in the examples, this is a fraction of conifer particles which has a high content of dihydroquercetin. This fraction of bark particles contains on average at least 50% by weight of all the dihydroquercetin contained in the starting bark material.

Moreover, as will be discussed below in the description, the inventors have also shown that the fraction (ii) of conifer bark particles having a particle size of equal to or less than a particle size ranging from 0.10 mm to 0.30 mm, for example a particle size of equal to or less than 0.16 mm, was enriched in other polyphenols, and specifically in tannins.

Similarly, in the method intended to obtain a composition enriched in tannins, the fraction(s) of conifer bark particles which are enriched in tannins, that is to say the content of tannins of which is substantially greater than the content of tannins of the conifer bark shavings used as starting material, are selected.

Additional Steps of the Method

The fraction of bark particles enriched in tannins obtained in step b) or b2) of the method may then be used for the purposes of obtaining purified compositions of tannins.

For example, a purified composition of tannins is obtained using known methods of extraction and purification using, as starting product, the fraction enriched in tannins resulting from step b) or b2) of the method described above.

Thus, according to some embodiments, the method described above comprises an additional step c) of obtaining a purified composition of tannins by extracting the tannins contained in the particles of the fraction enriched in tannins obtained in step b), or b2).

It is especially possible to use a method for solvent extraction of the tannins, using an aqueous-alcoholic solution or else using a water/acetone mixture. For the methods using a water/acetone solvent, the extraction step may be followed by a distillation step with the aim of eliminating the acetone, then a step for eliminating pigments and lipids by solvent, for example using the solvent hexane.

By way of illustration, to obtain a purified composition of tannins from the fraction of conifer bark particles selected in step b) of the method, those skilled in the art may carry out the extraction method which is described in application PCT no. WO 91/01989.

Similarly, the fraction of bark particles enriched in dihydroquercetin obtained in step b) of the method, or in step b2) in some variants of the method, may then be used for the purposes of obtaining purified compositions of dihydroquercetin.

For example, a purified composition of dihydroquercetin is obtained using known methods of extraction and purification using, as starting product, the fraction enriched in dihydroquercetin resulting from step b) or b2) of the method described above.

Thus, according to some embodiments, the method described above comprises an additional step c) of obtaining a purified composition of dihydroquercetin by extracting the dihydroquercetin contained in the particles of the fraction enriched in dihydroquercetin obtained in step b), or b2).

By way of example, those skilled in the art may carry out, in step c), a method for obtaining dihydroquercetin such as the method described in European patent application no. EP 2 639 232 or else the method described by Wang et al. (2011, Food Chemistry, Vol. 126: 1178-1185).

In advantageous embodiments, step c) comprises the following steps:

c1) carrying out solvent extraction of the polyphenols contained in the fraction of conifer bark particles enriched in dihydroquercetin selected in step c) of the method, in order to obtain an extract of polyphenols, and

c2) purifying the dihydroquercetin from the extract of polyphenols obtained in step d1).

Step c1) may be carried out according to any known method.

In some embodiments, step c1) may comprise extraction of the polyphenols using an aqueous-alcoholic solvent, for example with a solvent having an ethanol/water weight ratio of 25/75, for example at a temperature of 40° C., as described for example in European patent application no. EP 2 639 232.

According to other embodiments, step c1) comprises extraction of the polyphenols using an aqueous solvent comprising an enzyme or a plurality of enzymes such as a cellulase, a pectinase and/or a beta-glucosidase, as described in the paper by Wang et al. (2002, cited above).

According to yet other embodiments, step c1) comprises extraction of the polyphenols using water at a high temperature, as described in U.S. Pat. No. 3,189,596. Nonetheless, the latter method is not preferred, due to the fact that a high temperature is liable to degrade a portion of the dihydroquercetin contained in the conifer bark particles.

In order to carry out step c1), those skilled in the art may of course use any other known method, such as the method described in the application PCT no. WO 2013/172693 or else the method described by Ma et al. (2012, Int J Mol Sci, Vol. 13(7): 8789-8804).

Step c2) may be carried out by any method known to those skilled in the art. Step d2) is advantageously a step of purification by chromatography; as described for example in European patent application no. EP 2 639 232 or in the paper by Wang et al. (2002, cited above).

Any suitable type of chromatographic support may be used, such as reverse-phase chromatography supports (e.g. supports of the HiQ sil type sold by Chromatek, diol phase chromatography supports sold for example by YMC Europe or else C18 type supports sold by Kinetex). C18 type supports are preferred, especially the supports of this type sold by Kinetex.

Other Features of the Method

Depending on the genus of tree, the anatomical and physiological features of the bark tissues may vary considerably. It appears that conifers have a bark with a specific structure and composition, which has made it possible to develop the method for enriching in polyphenols, most particularly in tannins and in dihydroquercetin, which is defined in the present description.

As is shown in the examples, the tissues which constitute the bark of the Douglas pine have a structure and composition such that:
the tannins are concentrated in a particular tissue, ground into very fine particles, this fraction being denoted “F4” in the examples; and
the dihydroquercetin is found at high levels in a particular tissue located at the heart of the bark, which tissue has been denoted “T2” in the examples.

Advantageously, the method according to the invention is carried out using bark shavings from a conifer selected from cedar, cypress, pine, fir, juniper, agathis, larch, maritime pine (pinus pinaster), Douglas pine, and sequoia.

Shavings of Douglas pine (Pseudotsuga) are preferentially used for the method of obtaining a composition enriched in dihydroquercetin, and shavings of maritime pine (pinus pinaster) for the method of obtaining a composition enriched in tannins.

The present invention also relates to a fraction of bark particles enriched in tannins, obtainable by the method defined in the present description.

The invention also relates to a composition enriched in tannins, obtainable by the method defined in the present description.

The present invention also relates to a fraction of bark particles enriched in dihydroquercetin, obtainable by the method defined in the present description.

The invention also relates to a composition enriched in dihydroquercetin, obtainable by the method defined in the present description.

The compositions enriched in tannins or in dihydroquercetin may be used especially for the preparation of cosmetic compositions or pharmaceutical compositions.

It is known from the prior art that dihydroquercetin has properties of protecting against free radicals (antioxidant effect), antibacterial and antifungal properties, anti-inflammatory properties, properties of inhibiting deleterious effects caused by an excess of certain cytokines such as interferon-gamma, effects of increasing the amount of lipids in the skin, or else wound healing effects.

It is also known that dihydroquercetin especially has properties of protecting and reinforcing the capillary vascular network. Dihydroquercetin is especially useful in the treatment of vitamin deficiency, cardiac ischemia, atherosclerosis, diabetes mellitus, diseases of the liver, of the prostate, of the kidneys, of the gastrointestinal tract, of the urinary tract, cardiovascular diseases or else some cancers.

The composition enriched in dihydroquercetin may be used for preparing cosmetic compositions.

Tannins are mainly used for tanning leathers and hides, and also for dyeing certain fabrics. With regard to their use in health and cosmetics, it is known that tannins have antioxidant properties, which make them the compounds of choice for protection against the oxidative action of free radicals.

Method for Obtaining Compositions Enriched in Dihydroquercetin or in Tannins

As has been specified previously in the present description, step b), or b1), of the method for obtaining a composition based on conifer bark enriched in dihydroquercetin makes it possible to simultaneously obtain at least one fraction of conifer bark particles enriched in dihydroquercetin and at least one fraction of conifer bark particles enriched in tannins.

The inventors have shown that the fraction (ii) of conifer bark particles having a particle size of equal to or less than a particle size ranging from 0.10 mm to 0.30 mm, for example a particle size of equal to or less than 0.16 mm, which fraction is obtained in step b) of the method, was enriched in other polyphenols, and specifically was enriched in tannins.

Thus, in some embodiments of the method, step b), or b2), comprises selecting a fraction of bark particles enriched in tannins, said fraction consisting of bark particles having a size less than 0.30 mm, and preferentially of bark particles having a size less than 0.16 mm.

According to an alternative of these particular embodiments of the method, said method also comprises a step d) of obtaining a purified composition of tannins by extracting the tannins contained in the particles of the fraction enriched in tannins obtained in step b), or b2).

The invention also relates to a method for obtaining a composition based on conifer bark enriched in dihydroquercetin and a composition based on conifer bark enriched in tannins, comprising the following steps:

a) grinding dried shavings of bark from a conifer to obtain a ground particulate material of conifer bark,

b1) separating, depending on their size, the bark particles contained in the ground material obtained in step a) into a plurality of fractions comprising particles of distinct sizes, and

b2) selecting:
on the one hand, a fraction of bark particles enriched in dihydroquercetin, said fraction consisting of bark particles having a size greater than 0.6 mm, and preferentially of bark particles having a size greater than 1 mm, and
on the other hand, selecting a fraction of bark particles enriched in tannins, said fraction consisting of bark particles having a size less than 0.30 mm, and preferentially of bark particles having a size less than 0.16 mm.

It is possible to obtain purified compositions of dihydroquercetin from the fraction of particles enriched in dihydroquercetin obtained in step 3), according to known methods, to which reference is made elsewhere in the present description.

Thus, in some embodiments of the above method, said method also comprises a step 4) of obtaining a purified composition of tannins by extracting the tannins contained in the particles of the fraction enriched in tannins obtained in step 3).

It is also possible to obtain purified compositions of tannins from the fraction of particles enriched in tannins obtained in step 3), according to methods known to those skilled in the art, and especially according to the same type of methods which comprise at least one step of solvent extraction of the polyphenols and a step of purification, preferably by chromatography. It is especially possible to use a method for solvent extraction of the tannins, using an aqueous-alcoholic solution or else using a water/acetone mixture.

For the methods using a water/acetone solvent, the extraction step may be followed by a distillation step with the aim of eliminating the acetone, then a step for eliminating pigments and lipids by solvent, for example using the solvent hexane.

By way of illustration, to obtain a purified composition of tannins from the fraction of conifer bark particles selected in step 3) of the method, those skilled in the art may carry out the extraction method which is described in application PCT no. WO 91/01989.

The method of dry fractionation which is defined in the present description has numerous advantages, especially for carrying it out industrially. The method for obtaining compositions enriched in dihydroquercetin or in tannins may be effectively incorporated into industrial methods for the biorefinery of biomass and in particular of forestry, agricultural and industrial waste, and be compatible with an industrial economy aiming for sustainable development. Indeed, the method according to the invention comprises steps of enrichment in polyphenols of interest, respectively dihydroquercetin and tannins, which is carried out under dry conditions and which therefore does not require any consumption of water or consequently the requisite drying of effluents.

The present invention is also illustrated by the examples below, without being limited thereby.

### EXAMPLES

### Example 1: Method for Obtaining a Composition Enriched in Dihydroquercetin and a Composition Enriched in Tannins

A. Identification of the Tissues Constituting the Bark Having a High Content of Dihydroquercetin or Tannins

A first visual examination of a piece of Douglas pine bark makes it possible to distinguish three different tissues; a tissue T1 of light brown color, a tissue T2 of light beige color and a tissue T3 of darker beige color than the tissue T2, respectively.

The bark was manually dissected and the flavonoid composition of each tissue was determined by UPLC-MS analysis.

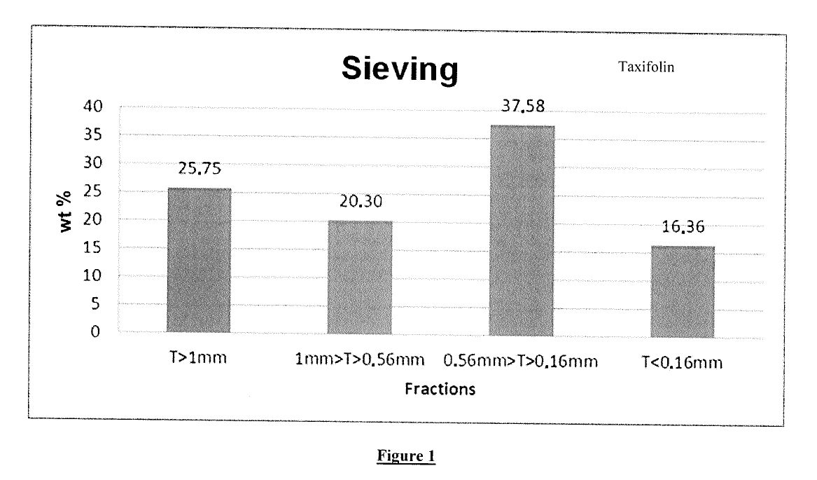

The results of the UPLC-MS analysis represented in FIG. 1 show that taxifolin is exclusively located in the tissue T2, while the tannins are located in the tissue T1.

These results justify the benefit in conceiving a method which makes it possible to selectively obtain (i) at least one fraction originating from bark enriched in taxifolin and (ii) at least one fraction originating from bark enriched in tannins.

B. Description of the Method

Douglas pine bark shavings from a logging operation carried out on Douglas pines in April 2013 were used.

The bark shavings were dried in an oven for 24 h at a temperature of 30° C. to 40° C. (this drying step is necessary to prevent clogging during grinding), then ground and separated by sieving.

The grinding was carried out in a Retsch SM 100 knife mill over a 6 mm grating. The mechanical stresses generated within a knife mill are mainly cutting and shearing.

The sample is conveyed by a hopper to the grinding chamber, within which a rotor (intermediate speed of rotation: 1500 rpm) fitted with knives ensures the movement of the sample and the grinding thereof between the rotor's own blades and the counter-rotating blades located in the chamber housing.

When the particles reach a size less than or equal to that of the mesh of the selection grating, they leave the grinding chamber and are discharged to the collecting container. The collected fraction was called F0. The residence time in the grinding chamber is relatively short, of the order of a few seconds.

The sieving operation consists in dividing or separating the ground particles by passing them through a succession of sieves, the grating meshes of which are increasingly small. On each sieve, the particles which are larger than the mesh are retained. Each fraction recovered corresponds to the diameter of the sieve which retained it. The sieving was carried out on a ROTEX station for 20 mins. The sieving was carried out on a sieving column fitted with several cylindrical trays with meshes of 1 mm, 0.56 mm and 0.16 mm, respectively, and also a lid preventing the loss of particles. The column is agitated by a vibration device, the speed of which can be controlled. Four fractions were obtained at the end of this sieving operation, denoted fractions F1 to F4.

C. Method for Quantifying the Taxifolin Present in the Different Fractions by UPLC-MS

Depolymerization of the Tannins Contained in Fractions F0-F4

Suspension A: fractions F0 to F4 (200 mg) were suspended in 2 ml of methanol.

Solution B: the depolymerization solution is prepared by adding 400 μl of thioglycolic acid to 4.96 ml of methanol containing 0.2 mol/l hydrochloric acid.

2 ml of the suspension A and 2 ml of the solution B are mixed in test tubes. The tube is sealed and the reaction mixture is heated at 90° C. for 6 min.

UPLC-ESI/MS Analyses

The liquid-phase chromatography system is an Acquity UPLC (Waters, Milford, Mass.) equipped with a diode array detector. The column (HSS 100×2 T3, 1 mm, 1.8 mm) is a Nucleosil 120-3 endcapped C18 (Macherey-Nagel, Sweden). The flow rate is 0.55 and the gradient conditions are as follows: solvent A (H₂O/HCOOH, 99/1, v/v); solvent B (CH₃CN/H₂O/HCOOH, 80/19/1, v/v/v); initial conditions 0.1% B; 0-5 min, 40% B linear; 5-7 min, 99% B linear; 7-8 min, 99% B and 8-9 min, 0.1% B linear.

The MS analyses were carried out on an AmaZon X ESI Trap mass spectrometer (Broker Daltonics, Bremen, Germany). In the source, the nebulizer pressure is 44 psi, the dry gas temperature was fixed at 200° C. with a flow rate of 12 l·min⁻¹ and the capillary voltage was set at 4.5 kV. The mass spectra were acquired over a mass range of 70-2000 Da in positive ionization mode. The rate of acquisition of the mass spectrum was fixed at 8.1 m/z·min⁻¹.

D. Method for Quantification of Tannins Present in the Different Fractions

The analysis and the quantification of the tannins are carried out after a depolymerization step.

The reaction for depolymerization of the tannins is a routine analytical method. The principle of this method is based on the acid-catalyzed breakage of the intermonomeric bonds. These bonds are broken heterolytically such that the extension units are released in the form of carbocations, trapped extemporaneously by the nucleophile (in this document the methyl ester of thioglycolic acid) present in large excess in the medium. Consequently, the distinction between extension units (which are detected in the form of thioether derivatives) and end units (which are released as they are, without any modification) makes it possible to estimate the mean degree of polymerization of the polymers present in the fraction. This depolymerization reaction may be carried out both on tannins, extracted beforehand from the plant matter or from a transformed product derived from the plant matter and directly on the plant material, in this instance the bark.

The depolymerization reaction was carried out directly on the powder in the five fractions F0, F1, F2, F3 and F4 studied. It was carried out in sealed tubes with acidified methanol (0.2 N with HCl) and brought to 90° C. in the presence of thioglycolic acid (which is converted into methyl ester during the preparation of the solution of the reagent) as nucleophilic agent.

E. Results of Analysis of the Fractions of Bark Particles Obtained According to the Method

At the end of the dry fractionation operations, four fractions, denoted F1 to F4, were collected:
Fraction F1: particles retained on the sieve of 1 mm
Fraction F2: particles retained on the sieve of 0.56 mm
Fraction F3: particles retained on the sieve of 0.16 mm
Fraction F4: particles passing through the 3 sieves

The fraction denoted F0 is the fraction recovered after grinding and before sieving.

The fractions collected were weighed and the percentage by weight of each fraction is represented in FIG. 1.

All analyses were carried out in triplicate in order to estimate the uncertainty interval of the measurement. The results of analysis and of quantification by UPLC-MS are collated in table 1 below.

TABLE 1
content of tannins and taxifolin in fractions F0 to F4.

| | tannins + | tannins/ | taxifolin | % | | |
|---|---|---|---|---|---|---|
| Sam- | taxifolin | g of | mg/g of | tannins + | % | % |
| ple | mg/g | bark | bark | taxifolin | tannins | taxifolin |
| F0 | 57.92322 | 31.63275 | 26.29047 | 5.8 | 3.2 | 2.6 |
| F1 | 75.28459 | 23.88729 | 51.3973 | 7.5 | 2.4 | 5.1 |
| F2 | 47.69746 | 33.29415 | 14.40331 | 4.7 | 3.3 | 1.4 |
| F3 | 41.09807 | 37.40727 | 3.690806 | 4.1 | 3.7 | 0.4 |
| F4 | 85.23012 | 79.01238 | 6.217749 | 8.5 | 7.9 | 0.6 |

The results from table 1 show that the Douglas pine barks contain 2.6% taxifolin. Moreover, carrying out the method according to the invention made it possible to isolate the majority (approximately 50%) of the total amount of taxifolin contained in the barks, which is contained in the fraction F1.

The results from table 1 also show that the condensed tannins are located in fraction F4. Thus, the method according to the invention may also be the means to extract the condensed tannins which may be used especially as precursors for epoxy resins based on a natural material.

All these results show that the method according to the invention makes it possible to simultaneously obtain (i) a fraction of conifer bark particles, specifically from Douglas pine, which is enriched in dihydroquercetin and (ii) a fraction of conifer bark particles, specifically from Douglas pine, which is enriched in tannins.

Observations with the naked eye made on the different fractions demonstrated that the different fractions (F1 to F4) consist of bark particles of different colors and compositions, implying different histological origins. The fraction F1 especially consists of three populations of particles:
F1a, fibrous, light in color,
F1 b solid, brown in color,
F1c solid, lighter in color.

UPLC-MS analysis, after depolymerization, of these three sub-fractions, reveals that the sub-fraction F1c contains the most taxifolin (table 2).

TABLE 2
content of tannins and taxifolin in fractions F1a to F1c.

| | Initial | | | | | | | | | |
|---|---|---|---|---|---|---|---|---|---|---|
| | weight | | | | | mg tannins | | % | | |
| | of bark | Initial | | | mg tannins/ | (−taxifolin)/ | mg taxifolin/ | tannins + | % | % |
| | (mg) | concentration | Dilution | dp | g of bark | g of bark | g of bark | taxifolin | tannins | taxifolin |
| F1a | 101.29 | 100 mg/ | 20x | 4.036774624 | 18.53785371 | 14.39254594 | 4.14530777 | 1.85 | 1.44 | 0.41 |
| | | 2 ml | | | | | | | | |
| F1b | 100.4 | 100 mg/ | 20x | 3.890543481 | 15.21557538 | 38.48271625 | 12.73285912 | 5.12 | 3.85 | 1.27 |
| | | 2 ml | | | | | | | | |
| F1c | 100.66 | 100 mg/ | 20x | 4.469119261 | 83.74925094 | 20.14746853 | 63.6017824 | 8.37 | 2.01 | 6.36 |
| | | 2 ml | | | | | | | | |

### Example 2: Reproducibility of the Method for Obtaining a Composition Enriched in Dihydroquercetin and a Composition Enriched in Tannins

The method described in example 1 was reproduced on Douglas pine barks.

The pine barks were ground in a knife mill then sieved through the series of sieves selected previously (sieves of 1 mm, 0.56 mm, 0.16 mm). The results obtained are presented in table 3.

TABLE 3
Comparison of the yields and content of tannins and taxifolin
obtained during the tests carried out in example 1 and in the present example 2.

| | | | % tannin + | | | | | | | |
|---|---|---|---|---|---|---|---|---|---|---|
| | Yield of | | taxifolin | | | | | | % taxifolin/ | |
| | each of | | in the | | % | | % | | total tannin + | |
| | the fractions | | bark | | tannin | | taxifolin | | taxifolin | |
| | Ex 1 | Ex 2 | Ex 1 | Ex 2 | Ex 1 | Ex 2 | Ex 1 | Ex 2 | Ex 1 | Ex 2 |
| F0 | | | 5.8% | | 3.2% | | 2.6% | | | 44.8% |
| F1 | 25.7% | 39.6% | 7.5% | 9.2% | 2.4% | 4.2% | 5.1% | 5% | 68.0% | 54.4% |
| F2 | 20.3% | 24.5% | 4.7% | 1.8% | 3.3% | 1.5% | 1.4% | 0.3% | 29.8% | 16.4% |
| F3 | 37.6% | 27.8% | 4.1% | 2.3% | 3.7% | 2.1% | 0.4% | 0.2% | 9.7% | 8.54% |
| F4 | 16.4% | 8% | 8.5% | 5.7% | 7.9% | 5.3% | 0.6% | 0.4% | 7.1% | 10.0% |

The results from table 3 demonstrate quite significant differences in yield over the two series of tests, this difference possibly being explained by the differences in some operating conditions which are different from those of the main features of the method of the invention, in particular the conditions for supplying the grinding device.

This technical problem was solved by installing a regulated automated system for supplying the grinding device.

### Example 3: Implementing the Method with a Step of Grinding Carried Out by a Ball Mill

The samples originating from the grinding in the knife mill over the 6 mm grating, fraction F0, were subjected to a second step of grinding within a Faure type ball mill, in a 5 l jar. 1 kg of dried pine bark shavings were introduced with 2 kg of balls (⅓ balls 1.2 cm in diameter, ⅓ balls 1.6 cm in diameter and ⅓ balls 2.6 cm in diameter) with a rotation speed of 120 rpm.

Samples were taken after 75 min and 12 h of grinding, and the samples were sieved over 1 mm, 0.56 mm and 0.16 mm gratings.

The contents of taxifolin and tannins were determined in the fraction of particles greater than 1 mm in size and in the fraction of particles less than 0.16 mm in size, having the highest contents of taxifolin and tannins, respectively (table 4).

TABLE 4
Yield and content of taxifolin obtained during purification by ball mill and sieving

| | | | % tannin + | | | | | | % taxifolin/ | |
|---|---|---|---|---|---|---|---|---|---|---|
| | | | taxifolin in | | | | | | total tannin + | |
| | Yield of | | the bark | | % tannin | | % taxifolin | | taxifolin | |
| | each of | | F0 | | | | | | | |
| | the fractions | | 5.8% | | 3.2% | | 2.6% | | 44.8% | |
| | 75 min | 12 h | 75 min | 12 h | 75 min | 12 h | 75 min | 12 h | 75 min | 12 h |
| x > 1 mm | 32.2% | 18.6% | 7.8% | 6.2% | 2.4% | 1.2% | 5.4% | 5% | 69.4% | 80.4% |
| 0.56 mm < | 17.1% | 21.6% | 2.1% | 2.4% | 1.3% | 1.6% | 0.8% | 0.8% | 38.0% | 33.3% |
| x < 1 mm | | | | | | | | | | |
| 0.16 mm < | 34.6% | 50.2% | 1.4% | 1.9% | 1.3% | 1.6% | 0.1% | 0.3% | 7.1% | 15.8% |
| x < 0.56 mm | | | | | | | | | | |
| X < | 16% | 9.7% | 4% | 3.5% | 3.7% | 3.2% | 0.3% | 0.3% | 7.5% | 8.7% |
| 0.16 mm | | | | | | | | | | |

The results of these tests demonstrate a very high concentration of taxifolin in the fraction of particles greater than 1 mm in size, which made it possible to isolate more than 80% of the taxifolin from the total phenols in the case of 12 h of grinding.

This result shows that it is possible, by a method which is easy to implement and inexpensive, to obtain final products with a high degree of purity in terms of taxifolin or tannins.

### Example 4: Method for Obtaining a Composition Enriched in Tannins

The method described in example 1 was reproduced on commercial maritime pine barks.

The step of grinding gave the fraction F0-M, the sieving of which gave 4 fractions: F1-M, F2-M, F3-M and F4-M.

As indicated in FIG. 1, the fractions F1 to F4 are characterized by the following particle sizes:
F1: particle size greater than 1 mm
F2: particle size less than 1 mm and greater than 0.56 mm
F3: particle size less than 0.56 mm and greater than 0.16 mm
F4: particle size less than 0.16 mm.

The contents of tannins and taxifolin of the 5 abovementioned fractions are presented in table 5.

TABLE 5
Phenolic composition of the fractions generated after grinding and
sieving the maritime pine barks.

| | Yield of | | | | | | |
|---|---|---|---|---|---|---|---|
| | each of | tannins + | tannins | taxifolin | % | | |
| | the | taxifolin | mg/g of | mg/g of | tannins + | % | % |
| Fraction | fractions % | mg/g | bark | bark | taxifolin | tannins | taxifolin |
| F0-M | 100% | 38.70669 | 38.46849 | 0.23819 | 3.87 | 3.85 | 0.02 |
| F1-M | 40.4% | 30.24864 | 29.96348 | 0.28515 | 3.02 | 3.00 | 0.02 |
| F2-M | 27.1% | 28.50223 | 28.24776 | 0.25447 | 2.85 | 2.82 | 0.03 |
| F3-M | 20.8% | 41.04847 | 40.72690 | 0.32156 | 4.10 | 4.07 | 0.03 |
| F4-M | 11.7% | 80.91985 | 80.57130 | 0.34854 | 8.09 | 8.05 | 0.04 |

The maritime pine is less rich in phenolic compounds than the Douglas pine. Moreover, unlike the Douglas pine, the amount of taxifolin in the maritime pine is further reduced. On the other hand, the fraction F4-M which contains the smallest particles (<0.16 μm) is substantially enriched in tannins (yield=11.7%).

1. A method for obtaining, from conifer bark, a composition enriched in tannins, comprising the following steps:
a) grinding dried shavings of bark from a conifer to obtain a ground particulate material of conifer bark, and
b) selecting, from the ground particulate material obtained in step a), a fraction of bark particles of smallest size representing at most 40% by weight, relative to the total weight of the ground particulate material obtained in step a), wherein the fraction of bark particles of smallest size is enriched in tannins.

2. The method as claimed in claim 1, wherein step b) comprises the following steps:
b1) separating, depending on size, bark particles contained in the ground particulate material obtained in step a) into a plurality of fractions comprising particles of distinct sizes, and
b2) selecting a fraction of bark particles enriched in tannins, said bark particles having a size less than 0.30 mm.

3. A method for obtaining, from conifer bark, a composition enriched in dihydroquercetin, comprising the following steps:
a) grinding dried shavings of bark from a conifer to obtain a ground particulate material of conifer bark, and
b) selecting, from the ground particulate material obtained in step a), a fraction of bark particles of largest size representing at most 50% by weight, relative to the total weight of the ground particulate material obtained in step a), wherein the fraction of bark particles of largest size is enriched in dihydroquercetin.

4. The method as claimed in claim 3, wherein step b) comprises the following steps:
b1) separating, depending on size, bark particles contained in the ground particulate material obtained in step a) into a plurality of fractions comprising particles of distinct sizes, and
b2) selecting a fraction of bark particles enriched in dihydroquercetin, said bark particles having a size greater than 0.6 mm.

5. The method as claimed in claim 3, further comprising selecting a fraction of bark particles enriched in tannins, said fraction having a size less than 0.30 mm.

6. The method as claimed in claim 1, wherein the dried shavings used in step a) have been previously subjected to a drying step in order to obtain dried shavings having a residual humidity equal to or less than 40%.

7. The method as claimed in claim 1, wherein step a) is carried out according to a method selected from (1) grinding in a knife mill, (2) grinding in a ball mill, and (3) grinding in a jet mill, or according to a combination of at least two of these methods.

8. The method as claimed in claim 1, wherein step b) is carried out by sieving.

9. The method as claimed in claim 1, further comprising a step c) of obtaining a purified composition of tannins by extracting the tannins contained in the bark particles of the fraction enriched in tannins obtained in step b).

10. The method as claimed in claim 3, further comprising a step c) of obtaining a purified composition of dihydroquercetin by extracting the dihydroquercetin contained in the bark particles of the fraction enriched in dihydroquercetin obtained in step b).

11. The method as claimed in claim 5, further comprising a step c) of obtaining a purified composition of tannins by extracting the tannins contained in the bark particles of the fraction enriched in tannins obtained in step b).

12. The method as claimed in claim 1, wherein said conifer is selected from the group consisting of cedar, cypress, pine, fir, juniper, agathis, larch, maritime pine, Douglas pine, and sequoia.

13. The method as claimed in claim 1, wherein said conifer is a maritime pine.

14. The method as claimed in claim 3, wherein said conifer is a Douglas pine.

15. A fraction of bark particles enriched in tannins, wherein the fraction is obtained by the method of claim 1; or
a composition enriched in tannins obtained from the fraction.

16. (canceled)

17. A fraction of bark particles enriched in dihydroquercetin, wherein the fraction is obtained by the method of claim 3, or
a composition enriched in dihydroquercetin obtained from the fraction.

18. (canceled)

19. The method of claim 2, wherein the bark particles have a size less than 0.16 mm.

20. The method of claim 4, wherein the bark particles have a size greater than 1 mm.

21. The method of claim 5, wherein the bark particles have a size less than 0.16 mm.

22. The method of claim 6, wherein the dried shavings have a residual humidity equal to or less than 15%.
